(19) United States
(12) Patent Application Publication
(10) Pub. No.: US 2004/0138120 A1
(43) Pub. Date: Jul. 15, 2004

(54) Hepatocyte growth factor variants

(75) Inventors: Daniel K. Kirchhofer, Los Altos, CA (US); Mark D. Peek, Vallejo, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/678,283

(22) Filed: Oct. 3, 2003

Related U.S. Application Data
(60) Provisional application No. 60/417,026, filed on Oct. 7, 2002.

(51) Int. Cl. A61K 38/18; C07K 14/475

(52) U.S. Cl. 514/12; 530/399; 435/69.1; 435/320.1; 435/325; 536/23.5

(57) ABSTRACT

Serine proteases factor XIa and kallikrein cleave and activate HGF. Cleavage is at two sites: a typical cleavage site, Arg494-Val495, and a novel cleavage site, Arg424-His425. Variant HGF fragments obtained by cleaving at one or both of these sites may be useful as agonists or antagonists of HGF. Variants or fragments obtained by modifying these cleavage sites so as to be resistant to kallikrein and/or FXIa cleavage are also provided.

### RELATED APPLICATIONS

This application is a non-provisional application filed under 37 CFR 1.53(b)(1), claiming priority under 35 USC 119(e) to provisional application No. 60/417,026 filed Oct. 7, 2002, the contents of which are incorporated herein by reference.

### BACKGROUND OF THE INVENTION

Hepatocyte growth factor (HGF)¹, the ligand for the tyrosine kinase receptor c-Met, was originally identified as a soluble factor with mitogenic activity for hepatocytes (1-4) and ‘scattering’ activity for epithelial cell colonies (5). The HGF/c-Met pathway is involved in many biological processes, such as embryonal development (6,7), angiogenesis (8), tissue regeneration and tumorigenesis (reviewed by (9,10)). The biologically active HGF is a disulfide-linked heterodimeric protein of ˜90 kDa consisting of an α-and β-chain (11). The α-chain is composed of an N-terminal PAN module (12) and four Kringle domains (K1-K4), whereas the β-chain has strong homology to the protease domain of serine proteases. What separates HGF from functionally active serine proteases are the changed residues Gln534 (instead of His) and Tyr673 (instead of Ser) which are part of the catalytic triad His-Asp-Ser of serine proteases. ¹Abbreviations used are: HGF, hepatocyte growth factor; K4, Kringle domain 4; uPA, urokinase-type plasminogen activator; FXIa, activated factor XIa; FXIIa, activated factor XIIa; HGFA, hepatocyte growth factor activator; APPI, Alzheimer's amyloid β-protein precursor inhibitor; HNC buffer, 20 mM Hepes, 150 mM NaCl, 5 mM CaCl₂ pH 7.5.

HGF is secreted into the extracellular matrix as a single chain form (pro-HGF) that lacks biological activity (13-17). It requires proteolytic cleavage at the Arg494-Val495 peptide bond to convert it into the active α/β heterodimer. Therefore, pro-HGF converting proteases constitute an important regulatory system in the HGF/c-Met signaling pathway. Pro-HGF has strong structural similarity to macromolecular substrates of serine proteases, particularly to plasminogen that also contains several Kringle domains. It is therefore not surprising that all pro-HGF converting enzymes identified so far belong to this enzyme family. Urokinase-type plasminogen activator (uPA), a serine protease known for converting plasminogen into plasmin, was shown to also have pro-HGF converting activity (15,18). There is an important difference in respect to the enzyme kinetics underlying these two uPA mediated proteolytic processes. uPA acts as a typical catalyst in activating plasminogen, whereas it converts pro-HGF in an unusual reaction that results in the formation of a stable complex of uPA and the reaction product HGF (19). Because this reaction does not follow classic enzyme kinetics, the efficiency of HGF formation will be low, as it is limited by the absolute number of uPA molecules present. It was suggested that this type of pro-HGF activation may be involved in invasive tumor growth (19).

Other known pro-HGF converting enzymes include factor XIIa (FXIIa) (20), HGF-activator HGFA (20-23), and the recently identified membrane-bound serine protease matriptase (24). FXIIa and HGFA both circulate in blood as zymogens and have a high overall homology in their amino acid sequences. Both activators follow classical enzyme kinetics and efficiently cleave pro-HGF at enzyme:substrate ratios of less than 1/1000 (20). HGFA is the best described pro-HGF activator and was suggested to play a role in generating active HGF during tissue regeneration (25,26), morphogenesis (27,28) and tumorigenesis (29-31). A common feature of all known pro-HGF activators is that they also undergo proteolytic activation to become active enzymes, a process that is mediated by yet another set of proteases. Thus, the HGF/c-Met pathway appears highly regulated and, depending on the particular biological process, may involve different activating enzyme and inhibitor systems.

It would therefore be beneficial to identify additional proteases that might be involved in cleavage of HGF, and to obtain further information regarding the criteria for protease cleavage such as cleavage sites, etc. Such data would provide novel targets for design of therapeutic agents and strategies.

### SUMMARY OF THE INVENTION

Activated HGF binds and activates the HGF receptor c-Met, thereby stimulating known downstream effects of the c-Met receptor. Therefore, alterations in the activation of HGF and/or in the ability of wild type HGF to bind and/or activate the HGF receptor would be expected to interfere with the downstream effects of the c-met receptor. Variants and fragments of HGF have been postulated to have potential agonist or antagonist activities. For e.g., Nakamura has described an HGF fragment composed of the α chain of HGF that apparently has antagonist activity against c-met/HGF receptor. See U.S. Pub. No. 2002/0004480 A1.

In order to identify other potential pro-HGF converting enzymes, a panel of serine proteases was screened. Plasma kallikrein and factor XIa (FXIa) were found to efficiently activate pro-HGF. Processing of pro-HGF by FXIa and by kallikrein is unprecedented in that each enzyme cleaves pro-HGF at two sites, as opposed to the single cleavage reaction of other known activators. By use of fragment analysis, HGF mutants and c-Met activation assays, the second cleavage site was identified and its functional impact was assessed. The results suggest that enzymes involved in inflammation and blood coagulation also participate in HGF-dependent processes, such as vascular remodeling.

Accordingly, the present invention provides novel activators of HGF (plasma kallikrein and factor XIa (FXIa)), methods for activating HGF using serine proteases kallikrein and factor XIa (FXIa), as well as novel fragments and variants of HGF resulting from cleavage by and/or alteration of the novel protease cleavage sites described herein. The proteases kallikrein and factor XIa cleave HGF between amino acid residues Arg424-His425, which is a heretofore unknown protease cleavage site, in addition to the conventional cleavage site at Arg494-Val495, where these amino acids are numbered according to the sequence of human HGF, including the signal sequence. Identification of the novel proteases and cleavage site provides for novel methods of activating and/or regulating activation of HGF/c-met, and for generation of novel polypeptide/peptide fragments and variants that could serve as agonists or antagonists of HGF. Included are polypeptides (or peptides) generated by protease kallikrein and/or factor XIa (FXIa) cleavage of HGF or variants thereof as described herein, useful as agonists and antagonists of HGF activity. These polypeptides may be advantageous in being smaller in size than wild type HGF and/or HGF fragments that would be obtained by cleaving at only the previously known site or by cleaving using previously known HGF proteases. Variants/fragments of a smaller size may provide various advantages, for e.g. greater tissue/cell penetrance, greater bioavailability, better in vivo biodistribution and/or greater flexibility/amenability to manipulations that enhance therapeutic efficacy. Alteration of the Arg424-His425 cleavage site so as to be protease resistant (and therefore not cleaved at this site when administered in vivo) could result in a variant/fragment that is able to compete with activated wild type HGF for binding to c-met, but is less biologically active (for e.g., less effective or essentially ineffective in activating c-met).

In one aspect, the invention provides an isolated polypeptide comprising a fragment of HGF (where HGF has the meaning defined in greater detail below), wherein said fragment comprises residues 1 to 424 of HGF. In another aspect, the invention provides an isolated polypeptide comprising a fragment of HGF, wherein said fragment comprises residues 425 to 494 of HGF. In yet another aspect, the invention provides an isolated polypeptide comprising a fragment of HGF, wherein said fragment comprises residues 425 to 494 and all or a portion of the β chain of HGF. In one aspect, the invention provides an isolated polypeptide comprising two fragments of HGF, wherein a first fragment comprises residues 1 to 424 of HGF, and a second fragment comprises residues 425 to 494 of HGF (for e.g., the first and second fragment may be linked by a non-peptide bond such as a disulfide bond, or the first and second fragment may be located in non-adjacent positions in the polypeptide). In some embodiments, the HGF fragment(s) in a polypeptide of the invention is linked or fused to a heterologous sequence (i.e., not an HGF sequence). Non-limiting examples of a heterologous sequence include an immunoglobulin sequence (e.g., Fc or portion thereof), phage coat protein or portion thereof, affinity tag (e.g., His tag), dimerization domain sequence (e.g., leucine zipper). In some embodiments, the polypeptides of the invention consist essentially of an HGF fragment as described above. For e.g., these polypeptides may contain moieties that enhance the biological and/or therapeutic characteristics of the polypeptide, for e.g. as described herein (such as glycosylation, pegylation, etc.). In another example, these polypeptides may contain non-HGF sequences (where a “non-HGF sequence” is a sequence having less than 90% 80%, 70% or 60% sequence identity with a contiguous sequence of HGF). In other embodiments, the polypeptides of the invention consist of an HGF fragment as described above. For example, a polypeptide of the invention may consist of an HGF fragment having residues 1 to 424. In another example, a polypeptide of the invention may consist of an HGF fragment having residues 425 to 494. In yet another example, a polypeptide of the invention may consist of an HGF fragment having residues 1 to 424 and an HGF fragment having residues 425 to 494. In various embodiments of polypeptides of the invention, the polypeptide does not contain, other than the specified HGF fragment(s), any other substantial and/or functional HGF sequence. For example, these polypeptides would not contain any other sequence that is identical to a contiguous sequence of at least 5, 10, 15, 20 or 25 residues of HGF.

The invention also provides variants of HGF that are resistant to proteolytic cleavage by enzymes such as kallikrein and/or factor XIa (FXIa), and are not capable of conversion into the active, two (or three)-chain form of HGF. The variants are preferably stabilized in single-chain form by mutations in amino acids that form enzyme recognition sites for kallikrein and/or factor XIa (FXIa). Such variants include those having an amino acid alteration at or adjacent to amino acid positions Arg424 or Arg494 in wild type human hepatocyte growth factor.

The invention also provides nucleic acid sequences encoding polypeptides of the invention, for e.g. HGF variants that are resistant to kallikrein and/or factor XIa, as described above, useful fragments of such HGF variants, replicable expression vectors containing and capable of expressing such nucleic acid sequences in a transformed host cell, and transformed host cells containing such nucleic acid sequences.

The invention also provides methods and compositions useful for modulating disease states associated with dysregulation of the HGF/c-met signaling axis. Thus, in one aspect, the invention provides a method of modulating c-met activation in a subject, said method comprising administering to the subject an effective amount of a polypeptide of the invention, whereby c-met activation is modulated. In one aspect, the invention provides a method of treating a pathological condition (for e.g., a cancer or immune-related condition) associated with activation of c-met in a subject, said method comprising administering to the subject an effective amount of a polypeptide of the invention (for e.g., an antagonist polypeptide), whereby c-met activation is inhibited. In another aspect, the invention provides a method of treating a pathological condition (for e.g., a cancer or immune-related condition) associated with reduced or inadequate activation of c-met in a subject, said method comprising administering to the subject an effective amount of a polypeptide of the invention (for e.g., an agonist polypeptide), whereby c-met activation is increased or enhanced.

The HGF/c-met signaling pathway is involved in multiple biological and physiological functions, including, for e.g., cell proliferation and angiogenesis. Thus, in another aspect, the invention provides a method of inhibiting c-met activated cell proliferation, said method comprising contacting a cell, tissue and/or subject with a condition (for e.g., cancer) associated with abnormal cell proliferation with an effective amount of a polypeptide of the invention (for e.g., an antagonist polypeptide), whereby cell proliferation associated with c-met activation is inhibited. In another aspect, the invention provides a method of increasing or enhancing c-met activated cell proliferation, said method comprising contacting a cell, tissue and/or subject with a condition associated with reduced or inadequate cell proliferation with an effective amount of a polypeptide of the invention (for e.g., an agonist polypeptide), whereby cell proliferation associated with c-met activation is increased or enhanced. In yet another aspect, the invention provides a method of modulating angiogenesis, said method comprising administering to a cell, tissue, and/or subject with a condition (for e.g., cancer) associated with abnormal angiogenesis an effective amount of a polypeptide of the invention, whereby angiogenesis is modulated. In an embodiment wherein angiogenesis is to be decreased or inhibited, the polypeptide would be an antagonist polypeptide of the invention. In an embodiment wherein angiogenesis is to be increased or enhanced, the polypeptide would be an agonist polypeptide of the invention.

### BRIEF DESCRIPTION OF THE DRAWINGS

FIG. 1 is an electrophoretic gel showing activation of ¹²⁵I-labeled pro-HGF by plasma kallikrein, FXIa and FXIIa. ¹²⁵I-pro-HGF (0.05 mM) was incubated for 4 hours at 37° C. with various concentrations (2-fold dilution steps; 80 nM in lane 2 down to 0.6 nM in lane 9) of (a) kallikrein, (b) FXIa and (c) FXIIa. Lane 1 (0) is t=0. The reaction mixtures were analyzed by SDS-PAGE (reducing conditions) using a 4-20% gradient gel followed by exposure on X-ray films. Indicated are the positions of the α (˜64 kDa) and β chains (˜36 kDa and ˜39 kDa) that were produced by cleavage at the primary cleavage site Arg494-Val495. The additional α2 band (˜54 kDa) was specifically generated by kallikrein and FXIa. (d) is a graph showing quantification of pro-HGF conversion by measuring the disappearance of the radiolabeled ˜90 kDa pro-HGF band. open circles, plasma kallikrein; filled circles, FXIa; open diamonds, FXIIa. Molecular weight standards are shown as Mᵣ×10³.

FIG. 2 is an electrophoretic gel showing inhibition of pro-HGF activation by specific inhibitors of plasma kallikrein, FXIa and FXIIa. ¹²⁵I-labeled pro-HGF (0.05 mg/ml) was incubated for 4 hrs at 37° C. with (a) kallikrein (80 nM), (b) FXIa (80 nM) and (c) FXIIa (40 nM) in the presence of the specific kallikrein inhibitor KALI-DY (250 nM), FXIa inhibitor APPI (250 nM) and FXIIa inhibitor corn trypsin inhibitor (250 nM). The HGF fragments were analyzed by SDS-PAGE as described in FIG. 1. Lane 1, enzyme+buffer at t=0; lane 2, enzyme+buffer at t=4 hours; lane 3, enzyme+APPI, lane 4, enzyme+KALI-DY; lane 5, enzyme+corn trypsin inhibitor.

FIG. 3 is a schematic representation of a model of the Kringle 4 (K4) domain of HGF depicting the kallikrein and FXIa cleavage site Arg424-His425. The model was based on the crystal structure of the Kringle 1 (K1) domain of HGF (42). The figure shows the side chains of the P3-P1 residues (Leu422, His423, Arg424) and the P1′-P4′ residues (His425, Ile426, Phe427, Trp428). The arrow indicates the peptide bond (Arg424-His425), which is located in a loop that is flanked by the two disulfide bonds Cys412-Cys452 and Cys440-Cys464. The disulfide bond network (Cys residues are indicated by numbers) prevents the release of the C-terminal 70 residue fragment (His425-Arg494) from the α-chain after cleavage of the Arg424-His425 peptide bond.

The insert shows non-labeled pro-HGF (0.3 mg/ml) activated by plasma kallikrein (Kal) and FXIa (XIa) analyzed by SDS-PAGE under reducing (+DTT=dithiotreitol) and non-reducing (−DTT) conditions. This experiment shows that HGF remains an intact molecule despite cleavage in K4 domain. Molecular weight standards are shown as Mᵣ×10³.

FIG. 4 is an electrophoretic gel showing the processing of HGF(R494E) by plasma kallikrein (Kal), FXIa (XIa) and FXIIa (XIIa). HGF(R494E) (0.3 mg/ml) in which the normal cleavage site was changed (Arg to Glu), as well as wildtype pro-HGF (Pro-HGF-wt) (0.3 mg/ml) were incubated with the enzymes (40 nM FXIIa, 80 nM kallikrein and FXIa) for 4 hrs at 37° C. Reaction products were analyzed by SDS-PAGE (reducing conditions). Gels were stained with Simply Blue Safestain. Indicated are the presence (solid line) or absence (dotted line) of the HGF chains. The ‘long’ β-chain (residues 425-728) generated by cleavage at the K4 domain site is shown as β_{His}425. The bands labeled with asterisks are the light and heavy chains of FXIa. Molecular weight standards are shown as Mᵣ×10³.

FIG. 5 is an electrophoretic gel showing resistance of the double mutant HGF(R424A:R494E) to proteolytic cleavage by kallikrein (Kal), FXIa (XIa) and FXIIa (XIIa). This HGF mutant incorporated a change at the deduced second cleavage site in Kringle 4 domain (Arg424Ala), in addition to the Arg494Glu change at the normal cleavage site (see FIG. 4). HGF(R424A:R494E) (0.3 mg/ml) was incubated with high concentrations of enzymes (320 nM of kallikrein and FXIa, 80 nM FXIIa) and analyzed as described in FIG. 4. The bands labeled with asterisks are the light and heavy chains of FXIa. Molecular weight standards are shown as Mᵣ×10³.

FIG. 6 is an electrophoretic gel showing c-Met receptor phosphorylation by HGF generated by plasma kallikrein (HGF_{Kallikrein}), FXIa (HGF_{FXIa}) and FXIIa (HGF_{FXIIa}). Human A549 lung carcinoma cells were incubated for 15 min with increasing concentrations of HGF produced by digesting pro-HGF with the enzymes as described in ‘Experimental procedures’. Only a small portion of HGF_{Kallikrein} and HGF_{FXIa} was processed at the second cleavage site (Arg424-His425) in the K4 domain (as shown in FIG. 3 insert). c-Met receptor was immunoprecipitated from cell lysates with an anti-c-Met antibody and analyzed after SDS-PAGE and electroblotting. Top panel: receptor was detected with anti-c-Met antibody; Bottom panel: c-Met receptor phosphorylation was detected with an anti-phosphotyrosine antibody. Molecular weight standards are shown as Mᵣ×10³.

FIG. 7 is an electrophoretic gel showing c-Met receptor phosphorylation by Kringle 4 domain-cleaved HGF generated by FXIa (HGF_{FXIa}) and FXIIa (HGF_{FXIIa}). (a) HGF_{FXIa} was completely cleaved at the normal cleavage site (Arg494-Val495) and almost completely at the second, K4 domain cleavage site (Arg424-His425), as indicated by the strong α2 band. The band labeled with asterisk is the light chain of FXIa. (b) Phosphorylation of c-Met by HGF_{FXIa} and HGF_{FXIIa} was determined as described in FIG. 6. Molecular weight standards are shown as Mᵣ×10³.

### DETAILED DESCRIPTION OF THE PREFERRED EMBODIMENTS

Definitions

As used herein, the terms “hepatocyte growth factor”, “HGF” and “huHGF” refer to a (human) growth factor capable of specific binding to a receptor of wild-type (human) HGF, which growth factor typically has a structure with six domains (finger, Kringle 1, Kringle 2, Kringle 3, Kringle 4 and serine protease domains), but nonetheless may have fewer domains or may have some of its domains repeated if it still retains its qualitative HGF receptor binding ability. This definition specifically includes the delta5 huHGF as disclosed by Seki et al., Biochem. Biophys. Res. Commun., 172:321-327 (1990). For example, these terms refer, unless specifically or contextually indicated otherwise, to any native or variant (whether native or synthetic) HGF polypeptide that is capable of activating the HGF/c-met signaling pathway under conditions that permit such process to occur. The terms “hepatocyte growth factor” and “HGF” also include hepatocyte growth factor from any non-human animal species, and in particular rat HGF.

The terms “wild-type human hepatocyte growth factor”, “native human hepatocyte growth factor”, “wild-type huHGF”, and “native huHGF” refer to native sequence human HGF such as that encoded by the cDNA sequence published by Miyazawa, et al., Biochem. Biophys. Res. Comm. (1989), 163:967-973, or Nakamura et al., Nature (1989), 342:440-443, including its mature, pre, pre-pro, and pro forms, purified from natural sources, chemically synthesized or recombinantly produced. The sequences reported by Miyazawa et al, and Nakamura et al. differ in 14 amino acids. The reason for the differences is not entirely clear; polymorphism or cloning artifacts are among the possibilities. Both sequences are specifically encompassed by the foregoing terms as defined for the purpose of the present invention. In one embodiment, the terms encompass the sequence reported by Miyazawa et al. In another embodiment, the terms encompass the sequence reported by Nakamura et al. It will be understood that natural allelic variations exist and can occur among individuals, as demonstrated by one or more amino acid differences in the amino acid sequence of each individual. Amino acid positions in the variant huHGF molecules herein are indicated in accordance with the numbering of Miyazawa et al. 1989, supra.

The terms “HGF receptor” and “c-Met” when used herein refer to a cellular receptor for HGF, which typically includes an extracellular domain, a transmembrane domain and an intracellular domain, as well as variants and fragments thereof which retain the ability to bind HGF. The terms “HGF receptor” and “c-Met” include the polypeptide molecule that comprises the full-length, native amino acid sequence encoded by the gene variously known as p190^{MET}. The present definition specifically encompasses soluble forms of HGF receptor, and HGF receptor from natural sources, synthetically produced in vitro or obtained by genetic manipulation including methods of recombinant DNA technology. The HGF receptor variants or fragments preferably share at least about 65% sequence identity, and more preferably at least about 75% sequence identity with any domain of the human c-Met amino acid sequence published in Rodrigues et al., 1991, Mol. Cell, Biol. 11:2962-2970; Park et al., 1987, Proc, Natl. Acad, Sci. 84:6379-6383; or Ponzetto et al., 1991, Oncogene, 6:553-559.

The terms “amino acid” and “amino acids” refer to all naturally occurring L-α-amino acids. This definition is meant to include norleucine, ornithine, and homocysteine. The amino acids are identified by either the single-letter or three-letter designations.

The terms “agonist” and “agonistic” when used herein refer to or describe a molecule which is capable of, directly or indirectly, substantially inducing, promoting or enhancing HGF biological activity and/or HGF receptor activation.

The terms “antagonist” and “antagonistic” when used herein refer to or describe a molecule which is capable of, directly or indirectly, substantially counteracting, reducing or inhibiting HGF biological activity and/or HGF receptor activation.

The terms “(HGF) biological activity”, “biologically active”, “activity” and “active” refer to any mitogenic, motogenic, and/or morphogenic activities exhibited by wild-type human HGF. HGF biological activity may, for example, be determined in an in vitro or in vivo assay of hepatocyte growth promotion. Adult rat hepatocytes in primary culture have been extensively used to search for factors that regulate hepatocyte proliferation. Accordingly, the mitogenic effect of an HGF variant can be conveniently determined in an assay suitable for testing the ability of an HGF molecule to induce DNA synthesis of rat hepatocytes in primary cultures, for example. Human hepatocytes are also available from whole liver perfusion on organs deemed unacceptable for transplantation, pare-downs of adult livers used for transplantation in children, fetal livers and liver remnants removed at surgery for other indications. Human hepatocytes can be cultured similarly to the methods established for preparing primary cultures of normal rat hepatocytes. Hepatocyte DNA synthesis can, for example, be assayed by measuring incorporation of ^{3H}-thymidine into DNA, with appropriate hydroxyurea controls for replicative synthesis.

“Resistant HGF variants” of the invention are defined herein as having one or more amino acid mutation in the HGF amino acid sequence that disrupts, deletes, or alters the cleavage site on the HGF molecule for serine proteases Factor XIa and/or kallikrein. For example, such variants include those disrupting the cleavage site at Arg494-Val495 and/or at Arg424-His425, for example by substituting, deleting, or adding amino acids. Preferred is the substitution of Arg424 and/or Arg494 with a non-basic amino acid, preferably with a neutral amino acid such as Ala.

The terms “transformed (host) cell”, “transformant”, and “transformed” refer to the introduction of nucleic acid, for example, DNA, into a cell. The cell is termed a “host cell”. The introduced DNA is usually in the form of a vector containing an inserted piece of DNA. The introduced DNA sequence may be from the same species as the host cell or a different species from the host cell, or it may be a hybrid DNA sequence, containing some foreign and some homologous DNA. The words transformants and transformed (host) cells include the primary subject cell and cultures derived therefrom, without regard to the number of transfers. It is also understood that all progeny may not be precisely identical in DNA content, due to deliberate or inadvertent mutations. Mutant progeny that have the same function or biological property as screened for in the originally transformed cell are included.

The technique of “polymerase chain reaction” or “PCR”, as used herein, generally refers to a procedure wherein minute amounts of a specific piece of nucleic acid, RNA and/or DNA, are amplified as described in U.S. Pat. No. 4,683,195, issued 28 Jul. 1987 and in Current Protocols in Molecular Biology, Ausubel et al. eds., Greene Publishing Associates and Wiley-Interscience 1991, Volume 2, Chapter 15.

A “disorder” is any condition that would benefit from treatment with a polypeptide or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result. A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA, and several copies of the vector and its inserted (foreign) DNA may be generated. In addition, the vector contains the necessary elements that permit translating the foreign DNA into a polypeptide. Many molecules of the polypeptide encoded by the foreign DNA can thus be rapidly synthesized.

Construction of the HGF Variants

Any technique known in the art can be used to perform site-directed mutagenesis, for example, those as disclosed in Sambrook et al., 1989, Molecular Cloning: A Laboratory Manual, second edition, Cold Spring Harbor Laboratory Press, New York. For example, oligonucleotide-directed mutagenesis is one preferred method for preparing the HGF variants of this invention. This method, which is well known in the art [Adelman et al. 1983, DNA, 2:183; Sambrook et al., Supra], is particularly suitable for making substitution variants, and may also be used to conveniently prepare deletion and insertion variants.

As will be appreciated, the site-specific mutagenesis technique typically employs a phage vector that exists in both a single-stranded and double-stranded form. Typical vectors useful in site-directed mutagenesis include vectors such as the M13 phage, for example, as disclosed by Messing et al., Third Cleveland Symposium on Macromolecules and Recombinant DNA, Editor A. Walton, Elsevier, Amsterdam (1981). These phage are readily commercially available and their use is generally well known to those skilled in the art. Alternatively, plasmid vectors that contain a single-stranded phage origin of replication (Veira et al., 1987, Meth. Enzymol., 153: 3) may be employed to obtain single-stranded DNA.

The oligonucleotides are readily synthesized using techniques well known in the art such as that described by Crea et al., 1978, Proc. Nat'l. Acad. Sci. U.S.A., 75: 5765.

Mutants with more than one amino acid substituted may be generated in one of several ways. If the amino acids are located close together in the polypeptide chain, they may be mutated simultaneously using one oligonucleotide that codes for all of the desired amino acid substitutions. If however, the amino acids are located some distance from each other (separated by more than ten amino acids, for example) it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

Another method for making mutations in the DNA sequence encoding wild-type HGF or a variant molecule known in the art, involves cleaving the DNA sequence encoding the starting HGF molecule at the appropriate position by digestion with restriction enzymes, recovering the properly cleaved DNA, synthesizing an oligonucleotide encoding the desired amino acid sequence and flanking regions such as polylinkers with blunt ends (or, instead of polylinkers, digesting the synthetic oligonucleotide with the restriction enzymes also used to cleave the HGF encoding DNA, thereby creating cohesive termini), and ligating the synthetic DNA into the remainder of the HGF encoding structural gene.

PCR mutagenesis is also suitable for making the HGF variants of the present invention, for example, as described in U.S. Pat. No. 4,683,195 issued 28 Jul. 1987 and in Current Protocols in Molecular Biology, Ausubel et al., eds. Greene Publishing Associates and Wiley-Interscience, Volume 2, Chapter 15, 1991. While the following discussion refers to DNA, it is understood that the techniques also find application with RNA. The PCR technique generally refers to the following procedure. When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers is designed to overlap the position of the mutation and to contain the mutation; the sequence of the other primer must generally be identical to a stretch of sequence of the opposite strand of the plasmid, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR amplification using a primer pair like the one just described results in a population of DNA fragments that differ at the position of the mutation specified by the primer, and possibly at other positions, as template copying is somewhat error-prone. If the ratio of template to product material is extremely low, the vast majority of product DNA fragments incorporate the desired mutation(s). This product material is used to replace the corresponding region in the plasmid that served as PCR template using standard DNA technology. Mutations at separate positions can be introduced simultaneously by either using a mutant second primer or performing a second PCR with different mutant primers and ligating the two resulting PCR fragments simultaneously to the vector fragment in a three (or more)-part ligation.

The cDNA encoding the HGF variants of the present invention is inserted into a replicable vector for further cloning or expression. Suitable vectors are prepared using standard recombinant DNA procedures. Isolated plasmids and DNA fragments are cleaved, tailored, and ligated together in a specific order to generate the desired vectors.

After ligation, the vector with the foreign gene now inserted is transformed into a suitable host cell. The transformed cells are selected by growth on an antibiotic, commonly tetracycline (tet) or ampicillin (amp), to which they are rendered resistant due to the presence of tet and/or amp resistance genes on the vector. If the ligation mixture has been transformed into a eukaryotic host cell, transformed cells may be selected by the DHFR/MTX system. The transformed cells are grown in culture and the plasmid DNA (plasmid refers to the vector ligated to the foreign gene of interest) is then isolated. This plasmid DNA is then analyzed by restriction mapping and/or DNA sequencing. DNA sequencing is generally performed by either the method of Messing et al.,1981 Nucleic Acids Res., 9:309 or by the method of Maxam et al., 1980, Methods of Enzymology, 65:499.

Prokaryotes are the preferred host cells for the initial cloning steps of this invention. They are particularly useful for rapid production of large amounts of DNA, for production of single-stranded DNA templates used for site-directed mutagenesis, for screening many mutants simultaneously, and for DNA sequencing of the mutants generated. For expressing the HGF variants of the present invention eukaryotic hosts, such as eukaryotic microbes (yeast) and multicellular organisms (mammalian cell cultures) may also be used. Examples of prokaryotes, e.g. E. coli, eukaryotic microorganisms and multicellular cell cultures, and expression vectors, suitable for use in producing the HGF variants of the present invention are, for example, those disclosed in WO 90/02798 (published 22 Mar. 1990).

Cloning and expression methodologies are well known in the art and are, for example, disclosed in the foregoing published PCT patent application (WO 90/02798).

If mammalian cells are used as host cells, transfection generally is carried out by the calcium phosphate precipitation method as described by Graham and Van der Eb, 1978, Virology, 52:546. However, other methods for introducing DNA into cells such as nuclear injection, electroporation, or protoplast fusion are also suitably used.

If yeast are used as the host, transfection is generally accomplished using polyethylene glycol, as taught by Hinnen, 1978, Proc. Natl. Acad. Sci. U.S.A., 75:1929-1933.

If prokaryotic cells or cells that contain substantial cell wall constructions are used, the preferred method of transfection is calcium treatment using calcium as described by Cohen et al., 1972, Proc. Natl. Acad. Sci. U.S.A. 69:2110, electroporation, and the like.

The HGF variant preferably is recovered from the culture medium as a secreted protein, although it also may be recovered from host cell lysates when directly expressed without a secretory signal. When the variant is expressed in a recombinant cell other than one of human origin, the variant is thus completely free of proteins of human origin. However, it is necessary to purify the variant from recombinant cell proteins in order to obtain preparations that are substantially homogeneous as to protein. The culture medium or lysate is generally centrifuged to remove particulate cell debris.

The variant is then purified from contaminant soluble proteins, for example, by an appropriate combination of conventional chromatography methods, e.g. gel filtration, ion-exchange, hydrophobic interaction, affinity, immunoaffinity chromatography, reverse phase HPLC; precipitation, e.g. ethanol precipitation, ammonium sulfate precipitation, or, preferably, immunoprecipitation with anti-HGF (polyclonal or monoclonal) antibodies covalently linked to Sepharose. Due to its high affinity to heparin, HGF can be conveniently purified on a heparin, such as heparin-Sepharose column. One skilled in the art will appreciate that purification methods suitable for native HGF may require modification to account for changes in the character of HGF or its variants upon expression in recombinant cell culture.

As hereinabove described, huHGF contains four putative glycosylation sites, which are located at positions 294 and 402 of the α-chain and at positions 566 and 653 of the β-chain. These positions are conserved in the rat HGF amino acid sequence. Glycosylation variants are within the scope herein.

Glycosylation of polypeptides is typically either N-linked or O-linked. N-linked refers to the attachment of the carbohydrate moiety to the side-chain of an asparagine residue. The tripeptide sequences, asparagine-X-serine and asparagine-X-threonine, wherein X is any amino acid except proline, are recognition sequences for enzymatic attachment of the carbohydrate moiety to the asparagine side chain. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be involved in O-linked glycosylation. O-linked glycoslation sites may, for example, be modified by the addition of, or substitution by, one or more serine or threonine residue to the amino acid sequence of the HGF molecule. For ease, changes are usually made at the DNA level, essentially using the techniques discussed hereinabove with respect to the amino acid sequence variants.

Chemical or enzymatic coupling of glycosydes to the HGF variants of the present invention may also be used to modify or increase the number or profile of carbohydrate substituents. These procedures are advantageous in that they do not require production of the polypeptide that is capable of O-linked (or N-linked) glycosylation. Depending on the coupling mode used, the sugar(s) may be attached to (a) arginine and histidine, (b) free carboxyl groups, (c) free hydroxyl groups such as those of cysteine, (d) free sulfhydryl groups such as those of serine, threonine, or hydroxyproline, (e) aromatic residues such as those of phenylalanine, tyrosine, or tryptophan or (f) the amide group of glutamine. These methods are described in WO 87/05330 (published 11 Sep. 1987), and in Aplin and Wriston, 1981, CRC Crit. Rev. Biochem., pp. 259-306.

Carbohydrate moieties present on an HGF variant may also be removed chemically or enzymatically. Chemical deglycosylation requires exposure to trifluoromethanesulfonic acid or an equivalent compound. This treatment results in the cleavage of most or all sugars, except the linking sugar, while leaving the polypeptide intact. Chemical deglycosylation is described by Hakimuddin et al., 1987, Arch. Biochem. Biophys. 259, 52 and by Edge et al.,1981, Anal. Biochem, 119, 131. Carbohydrate moieties can be removed by a variety of endo- and exoglycosidases as described by Thotakura et al.,1987, Meth. Enzymol. 138, 350. Glycosylation is suppressed by tunicamycin as described by Duskin et al.,1982, J. Biol. Chem. 257, 3105. Tunicamycin blocks the formation of protein-N-glycosydase linkages.

Glycosylation variants of the amino acid sequence variants herein can also be produced by selecting appropriate host cells. Yeast, for example, introduce glycosylation, which varies significantly from that of mammalian systems. Similarly, mammalian cells having a different species (e.g. hamster, murine, insect, porcine, bovine or ovine) or tissue (e.g. lung, liver, lymphoid, mesenchymal or epidermal) origin than the source of the selectin variant, are routinely screened for the ability to introduce variant glycosylation. Covalent modifications of an HGF variant molecule are included within the scope herein. Such modifications are traditionally introduced by reacting targeted amino acid residues of the HGF variant with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays of the HGF variants, or for the preparation of anti-HGF antibodies for immunoaffinity purification of the recombinant glycoprotein. For example, complete inactivation of the biological activity of the protein after reaction with ninhydrin would suggest that at least one arginyl or lysyl residue is critical for its activity, whereafter the individual residues which were modified under the conditions selected are identified by isolation of a peptide fragment containing the modified amino acid residue. Such modifications are within the ordinary skill in the art and are performed without undue experimentation.

Derivatization with bifunctional agents is useful for preparing intramolecular aggregates of the HGF variants as well as for cross-linking the HGF variants to a water insoluble support matrix or surface for use in assays or affinity purification. In addition, a study of interchain cross-links will provide direct information on conformational structure. Commonly used cross-linking agents include 1,1-bis(diazoacetyl)-2-phenylethane, glutaraldehyde, N-hydroxysuccinimide esters, homobifunctional imidoesters, and bifunctional maleimides. Derivatizing agents such as methyl-3-[(p-azidophenyl)dithio]propioimidate yield photoactivatable intermediates, which are capable of forming cross-links in the presence of light. Alternatively, reactive water insoluble matrices such as cyanogen bromide activated carbohydrates and the systems reactive substrates described in U.S. Pat. Nos. 3,959,642; 3,969,287; 3,691,016; 4,195,128; 4,247,642; 4,229,537; 4,055,635; and 4,330,440 are employed for protein immobilization and cross-linking.

Certain post-translational modifications are the result of the action of recombinant host cells on the expressed polypeptide. Glutaminyl and aspariginyl residues are frequently post-translationally deamidated to the corresponding glutamyl and aspartyl residues. Alternatively, these residues are deamidated under mildly acidic conditions. Either form of these residues falls within the scope of this invention.

Other post-translational modifications include hydroxylation of proline and lysine, phosphorylation of hydroxyl groups of seryl or threonyl residues, methylation of the α-amino groups of lysine, arginine, and histidine side chains [T. E. Creighton, 1983, In: Proteins: Structure and Molecular Properties, W. H. Freeman & Co., San Francisco, pp. 79-86].

Other derivatives comprise the novel HGF variants of this invention covalently bonded to a nonproteinaceous polymer. The nonproteinaceous polymer ordinarily is a hydrophilic synthetic polymer, i.e. a polymer not otherwise found in nature. However, polymers that exist in nature and are produced by recombinant or in vitro methods are useful, as are polymers that are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol.

The HGF variants may be linked to various nonproteinaceous polymers, such as polyethylene glycol, polypropylene glycol or polyoxyalkylenes, for example in the manner set forth in U.S. Pat. Nos. 4,640,835; 4,496,689; 4,301,144; 4,670,417; 4,791,192 or 4,179,337.

Therapeutic Compositions

The resistant HGF variants as well as HGF fragments formed by cleavage by factor XIa and/or kallikrein, can be used to block and/or compete with the binding of wild-type HGF to its receptor. This binding permits the treatment of pathologic conditions associated with the activation of an HGF receptor, such as malignancies associated with chronic HGF receptor activation and/or HGF overexpression.

The compounds of the present invention can be formulated according to known methods to prepare pharmaceutically useful compositions, whereby the HGF product is combined in admixture with a pharmaceutically acceptable carrier. Suitable carriers and their formulations are described in Remington's Pharmaceutical Sciences, 16th ed., 1980, Mack Publishing Co., edited by Oslo et al. These compositions will typically contain an effective amount of the HGF variant, for example, from on the order of about 0.5 to about 10 mg/ml, together with a suitable amount of carrier to prepare pharmaceutically acceptable compositions suitable for effective administration to the patient. The variants may be administered parenterally or by other methods that ensure its delivery to the bloodstream in an effective form.

Compositions particularly well suited for the clinical administration of the HGF variants used to practice this invention include sterile aqueous solutions or sterile hydratable powders such as lyophilized protein. Typically, an appropriate amount of a pharmaceutically acceptable salt is also used in the formulation to render the formulation isotonic.

Dosages and desired drug concentrations of pharmaceutical compositions of this invention may vary depending on the particular use envisioned. A typical effective dose in rat experiments is about 250 μg/kg administered as an intravenous bolus injection. Interspecies scaling of dosages can be performed in a manner known in the art, for e.g. as disclosed in Mordenti et al., 1991, Pharmaceut. Res. 8, 1351 and in the references cited therein.

The following examples merely illustrate the best mode now contemplated for practicing the invention, but should not be construed to limit the invention.

### EXAMPLE 1

Materials and Methods

Reagents

Pro-HGF expressed in CHO cells in the absence of serum and purified by HiTrap Sepharose SP chromatography, was obtained from David Kahn (Genentech, Inc.). Plasma purified human FXIIa, human FXIa and human plasma kallikrein were purchased from Haematologic Technologies (Essex Junction, Vt.) and from Enzyme Research (South Bend, Ind.). Recombinant tissue-type plasminogen activator was obtained from Canio Refino (Genentech, Inc.). Recombinant HGFA, which was produced by using an insect cell expression system, was provided by Jennifer Stamos (Genentech, Inc.). Complement factor C1s was from Enzyme Research and thrombin, factor IXa and factor Xa from Haematologic Technologies. Relipidated human tissue factor and recombinant human factor VIIa were produced as described (32,33). The Kunitz domain inhibitors Alzheimer's amyloid β-protein precursor inhibitor (APPI) (34) and KALI-DY (35) were generously provided by Mark Dennis (Genentech, Inc.). Corn trypsin inhibitor was from Haematologic Technologies and the molecular weight markers used were SeeBlue Plus2 and MultiMark standards (Invitrogen, Carlsbad, Calif.).

¹²⁵I-Labelling of Pro-HGF

For ¹²⁵I-labelling, a 250μl solution of IODO-GEN (1,3,4,6-tetrachloro-3α, 6α-diphenylglycoluril) (Pierce Chemical Co., Rockford, Ill.) in chloroform (0.5 mg/ml) was placed into 5-ml borosilicate glass tubes. Solvent was evaporated at room temperature under a steady stream of nitrogen gas and the dried material stored in a dessicator until further use. Pro-HGF (700 μg) in 20 mM Hepes, 150 mM NaCl, 5 mM CaCl₂ pH 7.5 buffer (HNC buffer) was added to the dried IODO-GEN material. ¹²⁵I-Labeled sodium solution (NEN Life Sciences Inc., Boston, Mass.) was added (5 μCi/μg protein) and the reaction mixture was incubated on ice for 5 min with gentle swirling. The material was then applied onto a PD-10 column (Pharmacia, Uppsala, Sweden), which had been equilibrated with 20 column volumes of HNC. Fractions containing the ¹²⁵I-labelled pro-HGF were collected and pooled. The specific activity was 1.8 μCi/μg pro-HGF.

Pro-HGF Activation Assays

0.05 mg/ml of ¹²⁵I-labelled pro-HGF in HNC buffer was incubated with increasing concentrations (0.6 nM-80 nM) of kallikrein, FXIa and FXIIa at 37° C. After 4 hrs aliquots were removed and added to sample buffer (Bio-Rad Laboratories, Hercules, Calif.) with or without reducing agent dithiotreitol (BIO-Rad). After a brief heating, samples (approx. 10⁶ cpm/lane) were loaded onto a 4-20% gradient polyacrylamide gel (Invitrogen Corp., Carlsbad, Calif.). After electrophoresis, the dried gels were exposed on x-ray films (X-OMAT AR, Eastman Kodak Company, Rochester, N.Y.) for 10-20 min. Films were developed (Kodak M35A X-OMAT Processor), scanned (Umax S-12, Umax Data Systems, Inc., Fremont, Calif.) and further processed with Adobe V.6.0 Photoshop software (Adobe Systems Inc., San Jose, Calif.). The bands corresponding to pro-HGF were cut from the dried gels and the radioactivity measured on a gamma counter (Iso-Data 100 Series). The data were fit to a 4-parameter equation using Kaleidagraph software (Synergy Software, Reading, Pa.) and the disappearance of pro-HGF quantified by determining the enzyme concentration that produced 50% substrate conversion (EC₅₀).

For inhibitor experiments ¹²⁵I-labelled pro-HGF (0.05 mg/ml) in HNC buffer was activated by kallikrein (80 nM), FXIa (80 nM) or FXIIa (40 nM) in the presence of 250 nM inhibitor. The inhibitors used were the kallikrein-specific Kunitz domain inhibitor KALI-DY (35), the FXIa-specific Kunitz domain inhibitor APPI (34) and the FXIIa-specific inhibitor corn trypsin inhibitor (36). After 4 hrs the reaction was stopped and the inhibition of HGF conversion was analyzed by SDS-PAGE under reducing conditions as described.

In experiments with the HGF mutants R494E and R424A:R494E, 0.3 mg/ml of the HGF mutants or wildtype pro-HGF were incubated with kallikrein (80 nM), FXIa (80 nM) or FXIIa (40 nM) in HNC buffer for 4 hrs at 37° C. Reaction aliquots were then loaded onto 4-20% gradient gels and analyzed under reducing conditions as described. Gels were stained with Simply Blue Safestain (Invitrogen).

N-terminal Amino Acid Sequencing

Samples containing HGF were reduced in 20 μl of BioRad Laemmli sample buffer adjusted to pH 8.3 containing 10 mM dithiotreitol at 85° C. for 5 min. Alkylation was performed by the addition of 2 μl of 200 mM of N-isopropyliodoacetamide (Krutzsch et a. 1993) in methanol at 25° C. for 20 minutes. Proteins were separated on BioRad precast gels and electroblotted onto PE-Applied Biosystems Problott membranes in a BioRad Trans-Blot transfer cell using 10 mM 3-{Cyclohexylamino}-1-propanesulfonic acid, pH 11.0, 10 mM thioglycolic acid, 10% methanol as the transfer buffer for 1 hr at 250 mA constant current (37). The PVDF membrane was stained with 0.1% Coomassie Blue R-250 in 50% methanol for 0.5 min and destained with 10% acetic acid in 50% methanol for 2-3 minutes. The membrane was thoroughly washed with water and allowed to dry for storage at 0° C.

Pyroglutamate deblocking was performed with pyroglutamate aminopeptidase. The PVDF band containing HGF protein was treated with 1-2 ul of methanol and blocked with 200 ul of 0.5% Zwitergent 3-16 (Calbiochem) in 0.1% acetic acid on a shaker for 5 minutes. The protein band was washed with 0.5 ml water to remove all traces of Zwitergent. The protein was deblocked with 1 milliunit (mu) of Pyrococcus Furiosus pyroglutamate aminopeptidase (Panvera Corp., Madison, Wis.) in 30 μl of 50 mM sodium phosphate, 10 mM dithiotreitol, 1 mM EDTA, pH 7.0 at 90° C. for 1 hr. The protein band was dried in a SpeedVac and directly sequenced.

Automated protein sequencing was performed on PE-Applied Biosystems Procise 494A protein sequencers. The Procise sequencers were equipped with 6 mm diameter micro-cartridges and an on-line PTH analyzer. The coupling buffer was N-methylpiperidine in N-propanol and water (25:60:15) supplied by PE-Applied Biosystems or distilled in-house. Twenty-minute Edman cycles were used as described (38) with two modifications. A high pressure (3.0 PSI) delivery of coupling buffer was carried out for 20 seconds prior to all phenylisothiocyanate deliveries. Peaks were integrated with Chromperfect (Justice Innovation software) and sequence interpretation was performed on a DEC Alpha computer (39).

Results

Proteolytic Cleavage of Pro-HGF by Plasma Kallikrein and Coagulation Factor XIa

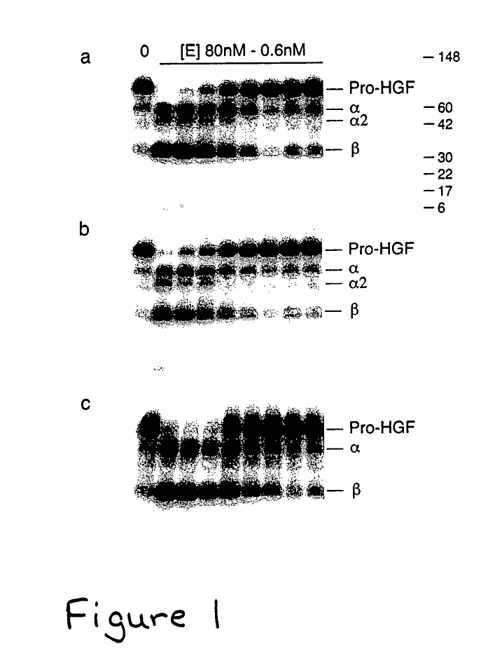

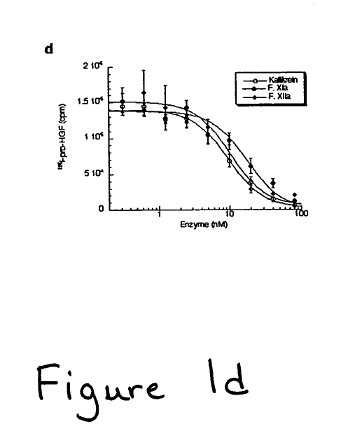

By use of purified ¹²⁵I-labelled pro-HGF the pro-HGF converting activity of a panel of serine proteases was examined. No pro-HGF processing activity was observed for complement factor C1s and the tissue factor/factor VIIa complex, nor for proteases previously examined by Shimomura et al. (20), such as factor Xa, thrombin, factor IXa and tissue-type plasminogen activator. However, plasma kallikrein (referred to as kallikrein throughout) and coagulation factor XIa (FXIa) each efficiently processed pro-HGF during a 4 hour reaction period (FIG. 1). Both enzymes cleaved pro-HGF at the normal cleavage site Arg494-Val495 thereby generating the α/β-chain heterodimer.

The N-termini of the HGF β-chains produced by each enzyme were identical (⁴⁹⁵VVNGIPTRTN⁵⁰⁴), the differences in mass of the two β-chains (˜36 kDa and ˜39 kDa) being attributed to differences in the content of attached carbohydrates (40). However, unlike the previously identified pro-HGF converting enzyme FXIIa (20), kallikrein and FXIa produced a second α-chain fragment (a2), whose apparent molecular mass of ˜54 kDa was about 10 kDa lower than the normal α chain (FIG. 1). Therefore, a ˜10 kDa fragment was released either from the N- or the C-terminus of the α chain. N-terminal sequencing showed that α2 and α chain N-termini were identical (data not shown), suggesting that the ˜10 kDa fragment arose by a cleavage at the C-terminal portion of the α-chain.

The pro-HGF converting activity of kallikrein and FXIa, quantified by measuring the disappearance of the ¹²⁵I-labelled HGF single chain, was similar to FXIIa. The concentrations of kallikrein, FXIa and FXIIa to convert 50% (EC₅₀) of pro-HGF during a 4 hr incubation period was 10 nM, 17 nM and 10 nM, respectively (FIG. 1d). The FXIa concentrations used throughout this study were of the naturally occurring homodimer (Mᵣ˜143,000) (41). Therefore, the EC₅₀ value based on monomeric FXIa concentration would be 34 nM.

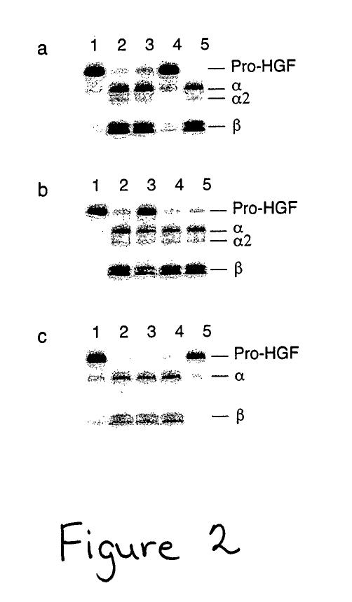

These findings were in apparent contradiction to the observed lack of pro-HGF converting activity of kallikrein and FXIa reported by Shimomura et al., 995 (20). In an attempt to understand these different results, we used inhibitors specific for kallikrein, FXIa and FXIIa to address the possibility of whether the pro-HGF converting activity in our assays could be due to contaminating proteases. The Kunitz domain inhibitors APPI (34) and KALI-DY (35) are potent and specific inhibitors of FXIa and kallikrein, respectively, while corn trypsin inhibitor is specific for FXIIa. FIG. 2 shows that KALI-DY only inhibited pro-HGF activation by plasma kallikrein, but not by FXIa and FXIIa. Conversely, APPI specifically interfered with FXIa-mediated pro-HGF activation, and corn trypsin inhibitor only inhibited FXIIa-mediated but not kallikrein- or FXIa-mediated pro-HGF activation (FIG. 2). Moreover, to rule out contaminating activity by the potent plasma derived pro-HGF activator, HGFA, we carried out an assay with recombinant HGFA. We found that neither KALI-DY nor APPI inhibited HGFA-dependent pro-HGF activation at concentrations (250 nM) that completely blocked pro-HGF activation by kallikrein and FXIa (data not shown). This experiment thus excluded HGFA as a possible contaminant. Furthermore, we employed kallikrein and FXIa preparations from two different commercial sources and used various buffer systems, only to find consistent and reproducible pro-HGF converting activities by these two enzymes. Therefore, we concluded that kallikrein and FXIa have the intrinsic ability to process pro-HGF.

### EXAMPLE 2

Site-Directed Mutagenesis, Expression and Purification of HGF Mutants

The HGF(R494E) mutant was previously described by Lokker et al. (17). Using HGF(R494E) as a template, Arg424 was altered to an Ala by site directed mutagenesis to give HGF(R424A:R494E) using the Muta-Gene mutagenesis kit (Bio-Rad Laboratories, Hercules, Calif.) according to manufacturer's protocol. The mutation was verified by DNA sequencing.

Recombinant proteins were produced using Chinese Hamster Ovary (CHO) cells in large scale transient transfection processes. Cells were grown in IL spinner flasks in F12/DMEM supplemented with Ultra-Low IgG serum (GibcoBRL) and Primatone HS (Sigma). The transfection process involved formation of the DNA-cationic lipid complex for 15 minutes in 300 ml of basal media followed by transfer of this complex to 700 ml of cell suspension (seeded at a density of 1.2×10⁶ cells/ml). The ratio of DNA to cationic lipid as well as the cell density were optimized to achieve maximal expression of recombinant protein. After 7 to 12 days the cell culture fluid was harvested and adjusted to 0.3M NaCl. The HGF mutants were purified by loading the cell culture fluid on a 5 mL HiTrap Sepharose SP chromatography column (Pharmacia, Uppsala, Sweden) pre-equilibrated with 20 mM Hepes pH 7.5, 0.3M NaCl. The column was washed with the same buffer and proteins were eluted with a gradient of 0.3M to 1.2M NaCl in 20 mM Hepes pH 7.5. The HGF-containing fractions were pooled, concentrated and the HGF concentration determined by quantitative amino acid analysis.

Results

Identification of the Alternative Kallikrein/FXIa Cleavage Site in the Kringle Domain 4 (K4)

The results described above (for e.g., Example 1) suggested that the unusual cleavage of the HGF α-chain by kallikrein and FXIa resulted in the release of a ˜10 kDa peptide upon reduction. Analyzing digested pro-HGF by reducing SDS-PAGE, a fragment of this size was identified and subjected to N-terminal sequencing. The sequence, ⁴²⁵HIFWEPDASK⁴³⁴, was consistent with the release of a 70-residue C-terminal α-chain fragment (His425-Arg494) of ˜10 kD as observed by SDS-PAGE. Therefore, cleavage probably occurred at the Arg424-His425 peptide bond in the K4 domain of the α-chain. Four testable predictions ensued from this assumption. First, since the putative cleavage site resided within a loop structure in K4 that is flanked by disulfide bonds, the cleaved HGF should migrate as a single band under non-reducing conditions. Second, the side chain of the P1 residue (Arg424) should be surface-exposed as it is required to occupy the specificity pocket of kallikrein and FXIa. Third, digestion of the primary cleavage site mutant HGF(R494E) (17) with kallikrein and FXIa should produce a ‘long’ β-chain having His425 as its N-terminal residue. Fourth, modification of the presumed K4 cleavage site should abolish proteolysis.

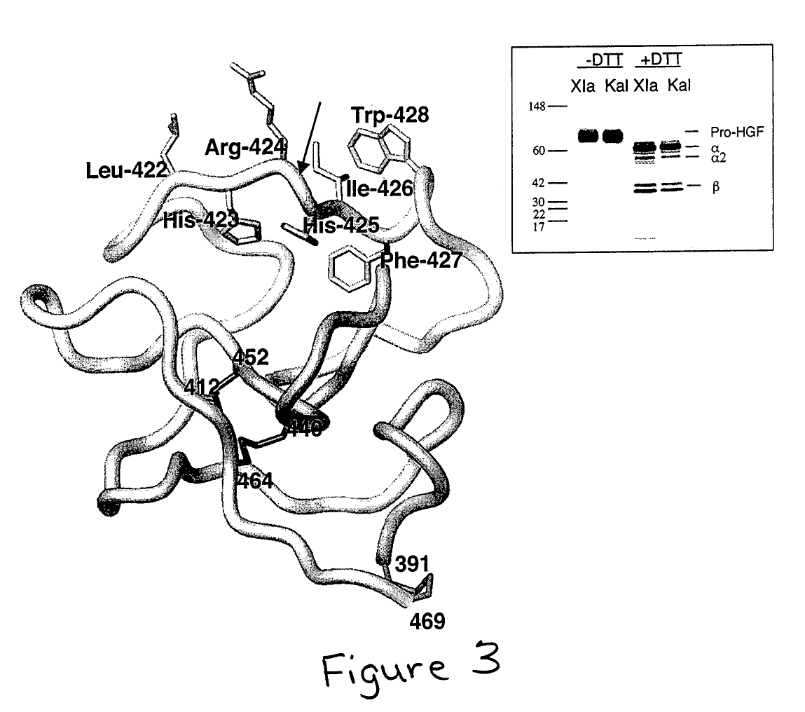

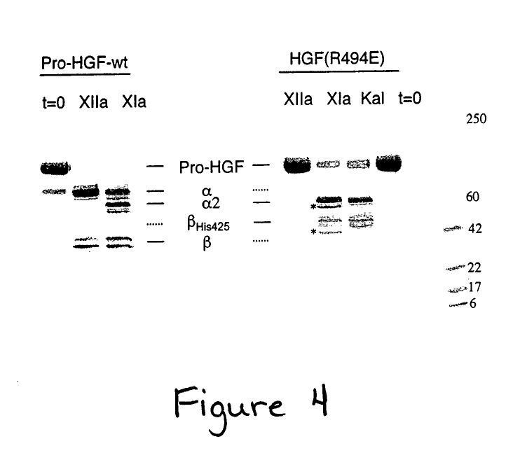

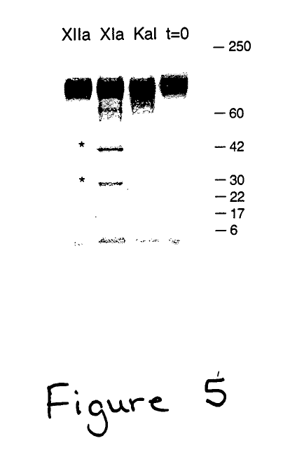

We attempted to systematically address these predictions. First, kallikrein- and FXIa-digested pro-HGF migrated as a single band of about 90 kDa on SDS gels under non-reducing conditions (FIG. 3, insert). This agreed with the hypothesis that the cleaved α-chain is held together by the disulfide bonds in K4. Second, a molecular model of K4 based on the crystal structure of K1 (42) was constructed (FIG. 3). In this model, the Arg424 side chain pointed away from the K4 backbone consistent with its accessibility for kallikrein and FXIa active sites. Third, the primary cleavage site mutant HGF(R494E) was processed by kallikrein and FXIa to produce the expected fragments, the α2-chain and the ‘long’ β-chain (FIG. 4), which had the N-terminal sequence ⁴²⁵HIFWEPDA⁴³². The reaction was specific in that the two Kunitz domain inhibitors KALI-DY and APPI inhibited the generation of these HGF fragments by kallikrein and FXIa, respectively (data not shown). Moreover, consistent with the mutation at the Arg494 residue, neither enzyme nor FXIIa were able to recognize this site for proteolysis anymore as indicated by the absence of the normal β chain bands (˜35 kDa and ˜38 kDa) on the gels (FIG. 4). The results also demonstrated that cleavage at Arg424-His425 was not contingent upon cleavage at Arg494-Val495. Fourth, a double mutant HGF(R424A:R494E) was constructed in which both P1 residues, Arg424 and Arg494, were changed. We found that kallikrein and FXIa were unable to process this mutant form anymore, even at high enzyme concentrations (FIG. 5).

### EXAMPLE 3

C-Met Phosphorylation Assay

Subconfluent A549 human lung carcinoma cells (ATCC, CCL-185) were serum-starved for 1 hr at 37° C. in DMEM:F12 (1:1). HGF (final concentrations of 25, 50, 100 and 200 ng/ml) activated by kallikrein, FXIa or FXIIa was added to the cells and incubated for 15 min at 37° C. After a rinse with cold Tris-buffered saline (50 mM Tris-HCl, pH 7.5, 150 mM NaCl), cells were lysed in Tris-buffered saline containing Ipegal CA630, protease inhibitor cocktail (Roche) and phosphatase inhibitor cocktail II (Sigma). Lysates were centrifuged at 10,000×g for 10 minutes, then the supernatants were treated with anti-c-Met antibody conjugated to agarose (SCBT, sc-161-AC from Santa Cruz Biotech, Calif.) for approximately 16 hours. The immunoprecipitates were separated by SDS-PAGE under reducing conditions, and electroblotted onto nitrocellulose membranes (Invitrogen, Carlsbad, Calif.). C-Met receptor was probed with anti-c-Met antibody (SCBT, sc-10; Santa Cruz Biotech, Calif.) followed by donkey anti-rabbit-HRP conjugate at 1:10,000 dilution (NA9340, Amersham). Phosphorylated c-Met was probed with an anti-phosphotyrosine antibody 4G10 (Upstate, Lake Placid, N.Y.) followed by sheep anti-mouse HRP conjugate at 1:2000 dilution. Antibody-bound proteins were detected with ECL Plus (Amersham).

Results

Phosphorylation of C-Met Receptor by Alternatively Cleaved HGF

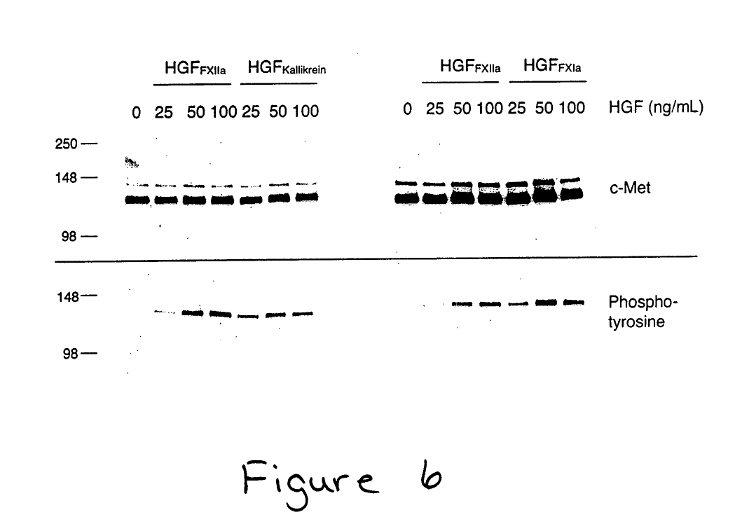

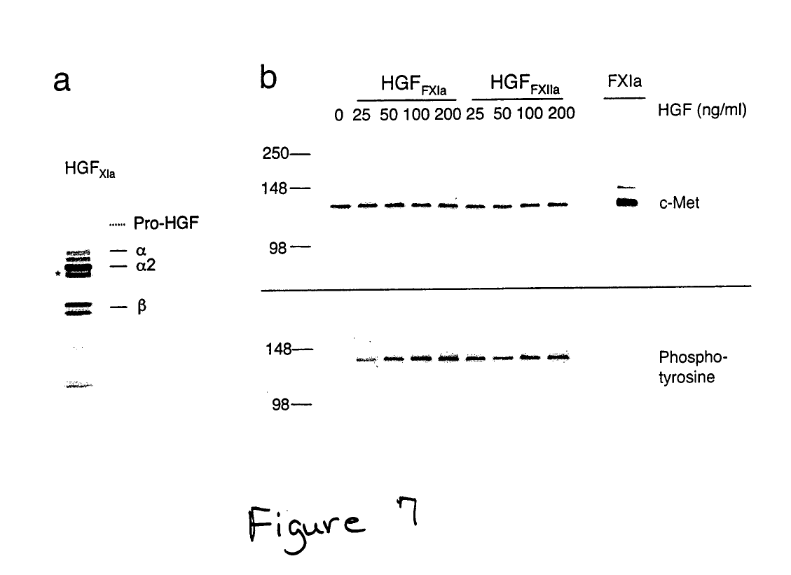

The activity of HGF generated by kallikrein (HGF_{Kallikrein}) and FXIa (HGF_{FXIa}) was assessed by measuring c-Met phosphorylation of A549 epithelial cells. FIG. 6 depicts the results obtained with HGF_{Kallikrein} an HGF_{FXIa} in which only a small portion of HGF was processed at the alternative K4 cleavage site, as exemplified by the HGF material shown in FIG. 3 (insert). HGF_{Kallikrein} and HGF_{FXIa} behaved like the reference HGF material generated by pro-HGF digestion with FXIIa and showed a concentration-dependent increase in c-Met phosphorylation activity. In most experiments the optimal c-Met phosphorylation activity was at concentrations 50-100 ng/ml HGF. Additional control experiments showed that the activators kallikrein and FXIa themselves had no activity in the assays (data not shown). To specifically address the question of whether cleavage at the K4 site (Arg424-His425) affected c-Met phosphorylation, high concentrations of FXIa were used to produce HGF in which K4 site cleavage approached near completion (FIG. 7a). This HGF form showed c-Met phosphorylation activity that was indistinguishable from the reference material (FXIIa-digested pro-HGF) (FIG. 7b) suggesting that cleavage at Arg424-His425 is without functional consequences in respect to c-Met activation as measured in this assay.

This application makes reference to numerous literature and patent citations. Each is hereby incorporated by reference for all purposes, as if fully set forth herein.

References

1. Nakamura, T., Nawa, K., and Ichihara, A. (1984) Biochem. Biophys. Res. Commun. 122, 1450-1459

2. Russell, W. E., McGowan, J. A., and Bucher, N. L. R. (1984) J. Cell. Physiol. 119, 183-192

3. Michalopoulos, G., Houck, K. A., Dolan, M. L., and Leutteke, N. C. (1984) Cancer Res. 44, 4414-4419

4. Nakamura, T., Teramoto, H., and Ichihara, A. (1986) Proc. Natl. Acad. Sci. U.S.A. 83, 6489-6493

5. Stoker, M., and Perryman, M. (1985) J. Cell Sci. 77, 209-223

6. Schmidt, C., Bladt, F., Goedecke, S., Brinkmann, V., Zschiesche, W., Sharpe, M., Gherardi, E., and Birchmeier, C. (1995) Nature (London) 373, 699-702

7. Uehara, Y., Minowa, O., Mori, C., Shiota, K., Kuno, J., Noda, T., and Kitamura, N. (1995) Nature (London) 373, 702-705

8. Grant, D. S., Kleinman, H. K., Goldberg, I. D., Bhargava, M. M., Nickoloff, B. J., Kinsella, J. L., Polverini, P., and Rosen, E. M. (1993) Proc. Natl. Acad. Sci. U.S.A. 90, 1937-1941

9. Trusolino, L., Pugliese, L., and Comoglio, P. M. (1998) FASEB J. 12, 1267-1280

10. van der Voort, R., Taher, T. E. I., Derksen, P. W. B., Spaargaren, M., van der Neut, R., and Pals, S. T. (2000) Adv. Cancer Res. 79, 39-90

11. Nakamura, T., Nishizawa, T., Hagiya, M., Seki, T., Shimonishi, M., Sugimura, A., Tashiro, K., and Shimizu, S. (1989) Nature (London) 342, 440-443

12. Tordai, H., Banyai, L., and Patthy, L. (1999) FEBS Lett. 461, 63-67

13. Gak, E., Taylor, W. G., Chan, A. M.-L., and Rubin, J. S. (1992) FEBS Lett. 311, 17-21

14. Naka, D., Ishii, T., Yoshiyama, Y., Miyazawa, K., Hara, H., Hishida, T., and Kitamura, N. (1992) J. Biol. Chem. 267, 20114-20119

15. Naldini, L., Tamagnone, L., Vigna, E., Sachs, M., Hartmann, G., Birchmeier, W., Daikuhara, Y., Tsubouchi, H., Blasi, F., and Comoglio, P. M. (1992) EMBO J. 11, 4825-4833

16. Hartmann, G., Naldini, L., Weidner, K. M., Sachs, M., Vigna, E., Comoglio, P. M., and Birchmeier, W. (1992) Proc. Natl. Acad. Sci. U.S.A. 89, 11574-11578

17. Lokker, N. A., Mark, M. R., Luis, E. A., Bennett, G. L., Robbins, K. A., Baker, J. B., and Godowski, P. J. (1992) EMBO J. 11, 2503-2510

18. Mars, W. M., Zarnegar, R., and Michalopoulos, G. K. (1993) Am. J. Pathol. 143, 949-958

19. Naldini, L., Vigna, E., Bardelli, A., Follenzi, A., Galimi, F., and Comoglio, P. M. (1995) J. Biol. Chem. 270, 603-611

20. Shimomura, T., Miyazawa, K., Komiyama, Y., Hiraoka, H., Naka, D., Morimoto, Y., and Kitamura, N. (1995) Eur. J. Biochem. 229, 257-261

21. Miyazawa, K., Shimomura, T., Kitamura, A., Kondo, J., Morimoto, Y., and Kitamura, N. (1993) J. Biol. Chem. 268, 10024-10028

22. Shimomura, T., Kondo, J., Ochiai, M., Naka, D., Miyazawa, K., Morimoto, Y., and Kitamura, N. (1993) J. Biol. Chem. 268, 22927-22932

23. Mizuno, K., Taneoue, Y., Okano, I., Harano, T., Takada, K., and Nakamura, T. (1994) Biochem. Biophys. Res. Commun. 198, 1161-1169

24. Lee, S.-L., Dickson, R. B., and Lin, C.-Y. (2000) J. Biol. Chem. 275, 36720-36725

25. Miyazawa, K., Shimomura, T., and Kitamura, N. (1996) J. Biol. Chem. 271, 3615-3618

26. Okajima, A., Miyazawa, K., Naitoh, Y., Inoue, K., and Kitamura, N. (1997) Hepatology 25, 97-102

27. Matsubara, Y., Ichinose, M., Yahagi, N., Tsukada, S., Oka, M., Miki, K., Kimura, S., Omata, M., Shiokawa, K., Kitamura, N., Kaneko, Y., and Fukamachi, H. (1998) Biochem. Biophys. Res. Commun. 253, 477-484

28. van Adelsberg, J., Sehgal, S., Kukes, A., Brady, C., Barasch, J., Yang, F., and Huan, Y. (2001) J. Biol. Chem. 276, 15099-15106

29. Hiscox, S., Davies, E. L., and Jiang, W. G. (1998) Br. J. Cancer 78, 150 (abstract)

30. Kataoka, H., Hamasuna, R., Itoh, H., Kitamura, N., and Koono, M. (2000) Cancer Res. 60, 6148-6159

31. Nagata, K., Hirono, S., Ido, A., Kataoka, H., Moriuchi, A., Shimomura, T., Hori, T., Hayashi, K., Koono, M., Kitamura, N., and Tsubouchi, H. (2001) Biochem. Biophys. Res. Commun. 289, 205-211

32. Presta, L., Sims, P., Meng, Y. G., Moran, P., Bullens, S., Bunting, S., Schoenfeld, J., Lowe, D., Lai, J., Rancatore, P., Iverson, M., Lim, A., Chisholm, V., Kelley, R. F., Riederer, M., and Kirchhofer, D. (2001) Thromb. Haemost. 85, 379-389

33. Dennis, M. S., Eigenbrot, C., Skelton, N. J., Ultsch, M. H., Santell, L., Dwyer, M. A., O'Connell, M. P., and Lazarus, R. A. (2000) Nature 404, 465-470

34. Dennis, M. S., and Lazarus, R. A. (1994) J. Biol. Chem. 269, 22129-22136

35. Dennis, M. S., Herzka, A., and Lazams, R. A. (1995) J. Biol. Chem. 270, 25411-25417

36. Hojima, Y., Pierce, J. V., and Pisano, J. J. (1980) Thromb. Res. 20, 149-162

37. Matsudaira, P. (1987) J. Biol. Chem. 262, 10035-10038

38. Henzel, W. J., Tropea, J., and Dupont, D. (1999) Anal. Biochem. 267, 148-160

39. Henzel, W. J., Rodriguez, H., and Watanabe, C. (1987) J. Chromatogr. 404, 41-52

40. Weidner, K. M., Behrens, J., Vandekerckhove, J., and Birchmeier, W. (1990) J. Cell Biol. 111, 2097-2108

41. Walsh, P. N. (2001) Thromb. Haemost. 86, 75-82

42. Ultsch, M., Lokker, N. A., Godowski, P. J., and de Vos, A. M. (1998) Structure 6, 1383-1393

43. Gailani, D., and Broze Jr., G. J. (1991) Science 253, 909-912

44. Seligsohn, U. (1993) Thromb. Haemost. 70, 68-71

45. Colman, R. W., and Schmaier, A. H. (1997) Blood 90, 3819-3843

46. Hathaway, W. E., Wuepper, K. D., Weston, W. L., Humbert, J. R., Rivers, R. P. A., Genton, E., August, C. S., Montgomery, R. R., and Mass, M. F. (1976) Am. J. Med. 60, 654-664

47. Rapaport, S. I., and Rao, L. V. M. (1992) Arterioscl. Thromb. 12, 1111-1121

48. Colman, R. W. (1999) Thromb.Haemost. 82, 1568-1577

49. Schmaier, A. H. (1997) Thromb. Haemost. 78, 101-107

50. Colman, R. W., Pixley, R. A., Najamunnisa, S., Yan, W., Wang, J., Mazar, A., and McCrae, K. R. (1997) J. Clin. Invest. 100, 1481-1487

51. Colman, R. W., Jameson, B. A., Lin, Y., Johnson, D., and Mousa, S. A. (2000) Blood 95, 543-550

52. Motta, G., Shariat-Madar, Z., Mahdi, F., Sampaio, C. A. M., and Schmaier, A. H. (2001) Thromb. Haemost. 86, 840-847

53. Gailani, D., Lasky, N. M., and Broze Jr., G. J. (1997) Blood Coag. Fibrinol. 8, 134-144

54. Weidner, K. M., Arakaki, N., Hartmann, G., Vandekerckhove, J., Weingart, S., Rieder, H., Fonatsch, C., Tsubouchi, H., Hishida, T., Daikuhara, Y., and Birchmeier, W. (1991) Proc. Natl. Acad. Sci. U.S.A. 88, 7001-7005

55. Pediaditakis, P., Monga, S. P. S., Mars, W. M., and Michalopoulos, G. K. (2002) J. Biol. Chem. 277, 14109-14115

56. Lay, A. J., Jiang, X.-M., Kisker, O., Flynn, E., Underwood, A., Condron, R., and Hogg, P. J. (2000) Nature (London) 408, 869-873

57. Stathakis, P., Fitzgerald, M., Matthias, L. J., Chesterman, C. N., and Hogg, P. J. (1997) J. Biol. Chem. 272, 20641-20645

58. Stathakis, P., Lay, A. J., Fitzgerald, M., Schlieker, C., Matthias, L. J., and Hogg, P. J. (1999) J. Biol. Chem. 274, 8910-8916

59. Date, K., Matsumoto, K., Kuba, K., Shimura, H., Tanaka, M., and Nakamura, T. (1998) Oncogene 17, 3045-3054

We claim:

1. A variant HGF protein comprising an amino acid modification that disrupts, deletes, or alters a cleavage site for serine protease Factor XIa or kallikrein corresponding to the cleavage site Arg424-His425 of human HGF.

2. The variant HGF of claim 1, further comprising a second amino acid modification that disrupts, deletes, or alters a cleavage site for serine protease Factor XIa or kallikrein corresponding to the cleavage site Arg494-His49.

3. The variant HGF of claim 1, wherein said modification comprises Arg424Ala.

4. The variant HGF of claim 2, wherein said second modification comprises Arg494Glu.

5. The variant HGF of claim 1, wherein said modification comprises substitution of Arg424 with a non-basic amino acid.

6. A nucleic acid sequence encoding the variant HGF of any of claims 1-5.

7. A replicable expression vector comprising the nucleic acid sequence of claim 6.

8. A host cell transformed with the vector of claim 7.

9. The nucleic acid sequence of claim 6, wherein the HGF protein is a human HGF protein.

10. A fragment of a hepatocyte growth factor protein consisting essentially of amino acids 1-424 of wild type human hepatocyte growth factor or variant thereof.

11. A nucleic acid sequence encoding the fragment of claim 10.

12. A replicable expression vector comprising the nucleic acid sequence of claim 11.

13. A host cell transformed with the vector of claim 12.

14. A nucleic acid sequence according to claim 11, wherein the hepatocyte growth factor is a human hepatocyte growth factor.

15. An HGF polypeptide consisting essentially of amino acids 1-424 in an alpha chain and amino acids 425-494 in an alpha chain, where the amino acids are numbered according to that of human HGF.

16. A nucleic acid sequence encoding the HGF polypeptide of claim 15.

17. A method for activating HGF comprising contacting said HGF with a serine protease selected from factor XIa or kallikrein.

18. An HGF polypeptide consisting essentially of amino acids 425 to 494 of human HGF or variant thereof.

19. A polypeptide comprising an HGF fragment having residues 1-424 of HGF fused to a heterologous sequence.

20. A polypeptide comprising an HGF fragment having residues 425-494 of HGF fused to a heterologous sequence.

21. A polypeptide comprising a first HGF fragment having residues 1-424 of HGF and a second HGF fragment having residues 425-494, wherein the first and second HGF fragments are linked through a non-peptide bond.

22. A polypeptide comprising a first HGF fragment having residues 1-424 of HGF and a second HGF fragment having residues 425-494, wherein the first and second HGF fragments are non-contiguous.

23. A polypeptide comprising an HGF fragment having residues 425-494 of an alpha chain fused to at least a portion of HGF β chain.

24. A polypeptide comprising an HGF fragment having residues 425-494 of an alpha chain linked through a non-peptide bond to at least a portion of HGF β chain.

25. A polypeptide consisting essentially of a first HGF fragment having residues 1-424 of HGF and a second HGF fragment having residues 425-494, wherein the first and second HGF fragments are linked through a non-peptide bond.

26. A polypeptide consisting essentially of a first HGF fragment having residues 1-424 of HGF and a second HGF fragment having residues 425-494, wherein the first and second HGF fragments are non-contiguous.

27. A polypeptide consisting essentially of an HGF fragment having residues 425-494 of an alpha chain fused to at least a portion of HGF β chain.

28. A polypeptide consisting essentially of an HGF fragment having residues 425-494 of an alpha chain linked through a non-peptide bond to at least a portion of HGF β chain.
